(19) United States
(12) Patent Application Publication
(10) Pub. No.: US 2008/0177129 A1
(43) Pub. Date: Jul. 24, 2008

(54) Therapeutic magnetic pulser device

(76) Inventor: Hsin Hsiang Pan, Taipei (TW)

Correspondence Address: SAM CHEN, 7F-1, 293, ROOSEVELT ROAD, SEC 3, TAIPEI, omitted

(21) Appl. No.: 11/834,681

(22) Filed: Aug. 6, 2007

(30) Foreign Application Priority Data
Jan. 23, 2007 (TW) 096201254

(51) Int. Cl. A61N 2/04

(52) U.S. Cl. 600/14

(57) ABSTRACT

A therapeutic magnetic pulser device is disclosed that includes a signal processing unit, a base frequency generating unit, a frequency display unit, a signal to pulse conversion unit, and a pulsed magnetic field generating unit. In one operation the signal processing unit is adapted to generate signals based on an output base frequency from the base frequency generating unit, a frequency of the signals is shown on the frequency display unit and the signals are sent to the signal to pulse conversion unit for converting the signals to pulses, and the pulses are fed to the pulsed magnetic field generating unit for generating pulsed magnetic fields which are capable of releasing pain and/or treating disease by applying electromagnetic energy to a person seated between north and south poles of the pulsed magnetic field generating unit.

### BACKGROUND OF THE INVENTION

1. Field of Invention

The invention relates to electromagnetic devices for alternative medicine and more particularly to a therapeutic magnetic pulser device with improved characteristics.

2. Description of Related Art

The effect of electromagnetic therapy is widely known by many people throughout the world. Such types of therapeutic magnetic pulser device are said to be capable of releasing pain and/or even treating disease by applying electromagnetic energy to the body.

For example, U.S. Pat. No. 4,665,898 discloses an apparatus which claims to eliminate tumor. Thus, continuing improvements in the exploitation of therapeutic magnetic pulser device are constantly being sought.

### SUMMARY OF THE INVENTION

It is therefore one object of the invention to provide a magnetic pulser device for releasing pain and/or treating disease by applying electromagnetic energy to the body.

The above and other objects, features and advantages of the invention will become apparent from the following detailed description taken with the accompanying drawings.

### BRIEF DESCRIPTION OF THE DRAWINGS

FIG. 1 is a schematic block diagram of a preferred embodiment of therapeutic magnetic pulser device according to the invention.

### DETAILED DESCRIPTION OF THE INVENTION

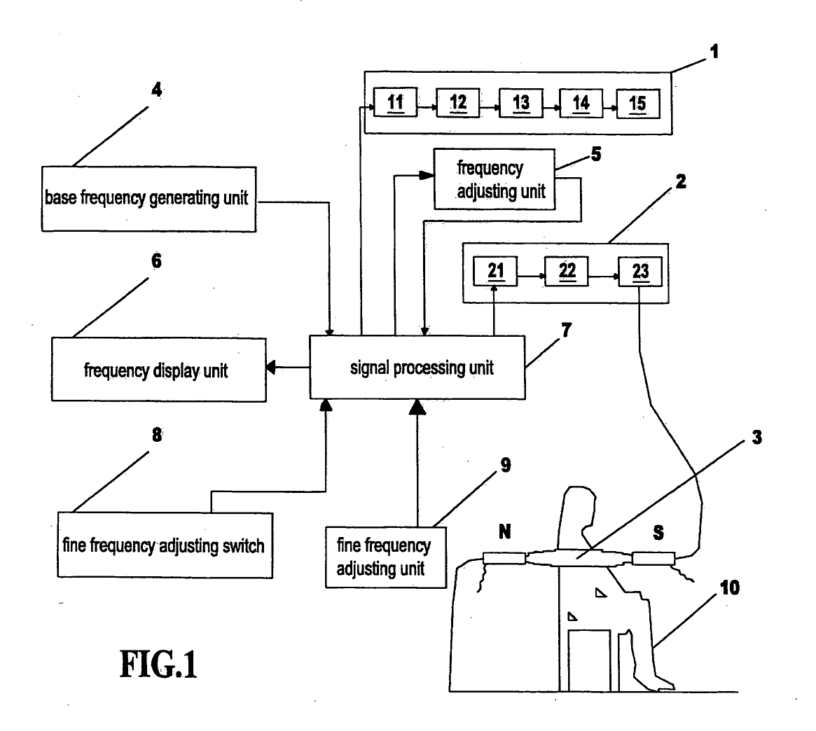

Referring to FIG. 1, a therapeutic magnetic pulser device in accordance with a preferred embodiment of the invention comprises a signal processing unit 7, a timing unit 1 electrically connected to the signal processing unit 7, a base frequency generating unit 4 electrically connected to the signal processing unit 7, a frequency display unit 6 electrically connected to the signal processing unit 7, a fine frequency adjusting switch 8 electrically connected to the signal processing unit 7, a fine frequency adjusting unit 9 electrically connected to the signal processing unit 7, a frequency adjusting unit 5 electrically connected to the signal processing unit 7, a signal to pulse conversion unit 2 electrically connected to the signal processing unit 7, and a pulsed magnetic field generating unit 3 electrically connected to the signal to pulse conversion unit 2. Each component and operations of the invention are discussed in detail below.

The pulsed magnetic field generating unit 3 is formed of coils. The signal processing unit 7 is adapted to generate signals based on an output base frequency from the base frequency generating unit 4 when the fine frequency adjusting switch 8 is open. The signals are then sent to the signal to pulse conversion unit 2. The signal to pulse conversion unit 2 comprises a signal amplifier 21, a signal to pulse conversion circuit 22, and a pulse amplification circuit 23 and thus is capable of converting signals to pulses with increased intensity. The pulses are fed to the pulsed magnetic field generating unit 3 for generating pulsed magnetic fields. The pulsed magnetic fields can release pain and/or treat disease of a person 10 seated between north and south poles of the pulsed magnetic field generating unit 3 by applying electromagnetic energy to the body of the person 10.

Note that the signals generated by the signal processing unit 7 have a frequency in the range of about 1 to about 1000 Hz. Also, the frequency is shown on the frequency display unit 6.

The timing unit 1 comprises a timer 11, a level converter 12, a signal converter 13, a power amplifier 14, and a buzzer 15. Thus, the timing unit 1 is adapted to audibly alert a user of the use time.

Alternatively, the fine frequency adjusting unit 9 is enabled when the fine frequency adjusting switch 8 is closed. The enabled fine frequency adjusting unit 9 is adapted to activate the frequency adjusting unit 5 which comprises a waveform switching control circuit (not shown) and a scale switching control circuit (not shown). Thus, the signal processing unit 7 is adapted to generate signals based on an output base frequency from the base frequency generating unit 4 and a fine adjustment signal from the frequency adjusting unit 5. Likewise, the signals are then sent to the signal to pulse conversion unit 2 which is adapted to convert signals to pulses with increased intensity. The pulses are fed to the pulsed magnetic field generating unit 3 for generating pulsed magnetic fields. The pulsed magnetic fields can release pain and/or treat disease of the person 10 seated between north and south poles of the pulsed magnetic field generating unit 3 by applying electromagnetic energy to the body of the person 10. Moreover, the signals generated by the signal processing unit 7 have a frequency in the range of about 1 to about 1000 Hz. Also, the frequency is shown on the frequency display unit 6.

The therapeutic magnetic pulser device further comprises an adapter (not shown) having a rectifier (not shown) for converting an external AC source into an operating DC power which is adapted to charge a rechargeable battery (not shown). The battery is adapted to supply operating power to the therapeutic magnetic pulser device in use.

While the invention herein disclosed has been described by means of specific embodiments, numerous modifications and variations could be made thereto by those skilled in the art without departing from the scope and spirit of the invention set forth in the claims.

What is claimed is:

1. A therapeutic magnetic pulser device comprising:
signal processing means;
base frequency generating means electrically connected to the signal processing means;
frequency display means electrically connected to the signal processing means;
signal to pulse conversion means electrically connected to the signal processing means; and
pulsed magnetic field generating means electrically connected to the signal to pulse conversion means,
wherein the signal processing means is adapted to generate signals based on an output base frequency from the base frequency generating means, a frequency of the signals is shown on the frequency display means and the signals are sent to the signal to pulse conversion means for converting the signals to pulses, and the pulses are fed to the pulsed magnetic field generating means for generating pulsed magnetic fields.

2. The therapeutic magnetic pulser device of claim 1, wherein the frequency of the signals is in a range of about 1 Hz to about 1000 Hz.

3. The therapeutic magnetic pulser device of claim 1, wherein the signal to pulse conversion means comprises a signal amplifier, a signal to pulse conversion circuit, and a pulse amplification circuit.

4. The therapeutic magnetic pulser device of claim 1, further comprising timing means electrically connected to the signal processing means, the timing means comprising a time, a level converter, a signal converter, a power amplifier, and a buzzer.

5. The therapeutic magnetic pulser device of claim 1, further comprising fine frequency adjusting means electrically connected to the signal processing means, a fine frequency adjusting switch electrically connected to the signal processing means, and frequency adjustment means electrically connected to the signal processing means, wherein the fine frequency adjusting means is enabled when the fine frequency adjusting switch is closed, and the fine frequency adjusting means is adapted to activate the frequency adjustment means so that the signal processing means is adapted to generate signals based on the output base frequency from the base frequency generating means and a fine adjustment signal from the frequency adjustment means.

6. The therapeutic magnetic pulser device of claim 5, wherein the frequency adjustment means comprises a waveform switching control circuit and a scale switching control circuit.
